(19) United States
(12) Patent Application Publication
(10) Pub. No.: US 2020/0286615 A1
(43) Pub. Date: Sep. 10, 2020

(54) METHOD FOR ANALYSING A MEDICAL IMAGING DATA SET, SYSTEM FOR ANALYSING A MEDICAL IMAGING DATA SET, COMPUTER PROGRAM PRODUCT AND A COMPUTER-READABLE MEDIUM

(71) Applicant: Siemens Healthcare GmbH, Erlangen (DE)

(72) Inventors: Andre HARTUNG, Erlangen (DE); Razvan IONASEC, Nuernberg (DE)

(73) Assignee: Siemens Healthcare GmbH, Erlangen (DE)

(21) Appl. No.: 16/650,501

(22) PCT Filed: Sep. 26, 2018
(86) PCT No.: PCT/EP2018/076147

§ 371 Date: Mar. 25, 2020

(30) Foreign Application Priority Data
Oct. 5, 2017 (EP) 17194971.2

(51) Int. Cl. G16H 30/40; G06T 7/00; G16H 15/00; G16H 50/20

(52) U.S. Cl. CPC G16H 30/40; G06T 7/0012; G16H 15/00; G06T 2207/30004; G06T 2207/20081; G06T 2207/20084; G16H 50/20

(57) ABSTRACT

A method for analysing a medical imaging data set is disclosed. In an embodiment, the method includes providing the medical imaging data set; assigning a probability value for a negative finding, in particular for a negative finding of a specific type of abnormality, to the medical imaging data set. The probability value is based on the image data set; and providing the medical imaging data set automatically either to an output device for analysing the medical imaging data set or to a device for storing the medical imaging data set based on the probability value.

### PRIORITY STATEMENT

This application is the national phase under 35 U.S.C. § 371 of PCT International Application No. PCT/EP2018/076147 which has an International filing date of Sep. 26, 2018, which designated the United States of America and which claims priority to European patent application no. EP 17194971.2 filed Oct. 5, 2017, the entire contents of each of which are hereby incorporated herein by reference.

### FIELD

Embodiments of the invention generally relate to a method for analysing a medical imaging data set, a system for analysing a medical imaging data set, a computer program product and a computer-readable medium.

### BACKGROUND

Medical imaging data sets are well known in the prior art. In general, medical imaging data sets are recorded and reconstructed by medical imaging devices, such as an X-ray scanner, computer tomography (CT) scanner, magnetic resonance tomography (MRT) scanner or ultrasound scanner, for example. However, after recording and reconstructing the medical imaging data set, a detailed analysis has to be performed by a specialist, for example by a radiologist, for identifying abnormality in the medical imaging data set. Thereby, a large proportion of these subsequent detailed analyses or exams leads to an irrelevant result due to an absence of any abnormality, i. e. a success rate of identifying an abnormality in a medical imaging data set is comparatively low. Nevertheless, those analyses or exams have to be performed, although the majority of the recorded medical imaging data sets have no indication for abnormalities. As a consequence, free capacities for analysing medical imaging data sets are reduced.

### SUMMARY

At least one embodiment of the present application provides a method for analysing medical imaging data sets, wherein an effort for analysing medical imaging data sets is reduced and/or a success rate for identifying an abnormality in a medical imaging data set is increased.

Embodiments of the present application are directed to a method, a system, a computer program product and a computer readable computer medium.

According to a first embodiment of the present application, a method for analysing a medical imaging data set is provided, comprising:

providing the medical imaging data set;

assigning a probability value for a negative finding to the medical imaging data set, wherein the probability value is based on the image data set; and

providing the medical imaging data set automatically either to an output device for analysing the medical imaging data set or to a device for storing the medical imaging data set based on the probability value and/or for creating a report data set-based on the probability value.

In at least one embodiment, the medical imaging data set is recorded by a medical imaging device such as a x-ray-scanner, a CT (computer tomography scanner)—scanner, a MRT (magnetic resonance tomography)—scanner or the like, and subsequently the medical imaging data set is provided, preferably to a control unit. The control unit comprises a processor being configured for executing at least:

providing the medical imaging data set automatically either
to an output device for analysing the medical imaging data set or
to a device for storing the medical imaging data set and/or for creating a report data set based on the probability value.

Another embodiment of the present invention is directed to a system for analysing a medical imaging data set comprising the medical instrument and a server, wherein the system is configured for:

recording the medical imaging data set by a medical imaging device;

providing a probability value for a negative finding, in particular for a negative finding of a specific type of abnormality, based on the medical imaging data set; and

providing the medical imaging data set automatically either
to an output device for analysing the medical imaging data set or
to a device for storing the medical imaging data set and/or for creating a report data set based on the probability value.

Another embodiment of the present invention is directed to a method for analysing a medical imaging data set, comprising:

providing the medical imaging data set;

assigning a probability value for a negative finding to the medical imaging data set, the probability value being based on the image data set; and

at least one of
providing the medical imaging data set automatically to at least one of an output device for analysing the medical imaging data set or to a device for storing the medical imaging data set, and creating a report data set based on the probability value.

In particular, at least one embodiment of the system comprises a control unit having a processor configured for executing at least one of the steps of at least one embodiment of the method described above.

Another embodiment of the present invention is directed to a computer program product for carrying out the steps of the method according to at least one embodiment of the present invention when the computer program product is loaded into a memory of a programmable device.

A further embodiment of the present invention is a computer-readable medium on which is stored a program elements that can be read and executed by a computer unit in order to perform steps of at least one embodiment of the method according to the present invention when the program elements are executed by the computer unit.

### BRIEF DESCRIPTION OF THE DRAWINGS

In the drawings:

FIG. 1 shows a block diagram illustrating a system for analysing a medical imaging data set according to a preferred embodiment of the present invention.

FIG. 2 shows a flow diagram illustrating a method for analysing a medical imaging data set according to a preferred embodiment of the present invention.

### DETAILED DESCRIPTION OF THE EXAMPLE EMBODIMENTS

According to a first embodiment of the present application, a method for analysing a medical imaging data set is provided, comprising:

providing the medical imaging data set;

assigning a probability value for a negative finding to the medical imaging data set, wherein the probability value is based on the image data set; and

providing the medical imaging data set automatically either
to an output device for analysing the medical imaging data set or
to a device for storing the medical imaging data set based on the probability value and/or for creating a report data set based on the probability value.

It is provided according to at least one embodiment of the present application, that the medical imaging data set is transferred either to an output device or to a device for storing the medical imaging data set and/or for creating a report data set based on the probability value, i. e. a preselection of the medical imaging data sets is performed. As a consequence, a specialist for analysing the medical imaging data sets in detail is no longer in charge of analysing those medical imaging data sets that have a high probability of identifying no abnormality in a detailed analysis.

The probability value preferably represents a probability for successively identifying no abnormality by analysing the medical imaging data set in detail. By skipping the analysis of those medical imaging data sets having a comparatively high probability for identifying no abnormality it is further advantageously possible to increase the success rate or concentrate the analysing effort to those analyses having a certain probability of being successful in identifying an abnormality.

In other words: the method according to at least one embodiment of the present application preselects medical imaging data sets and redirects the effort for analysing the medical imaging data sets to those having a low probability for a negative finding. Subsequently, the analysed medical imaging data sets are preferably transferred to the device for storing the medical imaging data set, too. As a result of the method a rate for providing analysed medical imaging data sets to the device for storing the medical imaging data sets per time is increased, advantageously, since a comparatively high number of medical imaging data sets are transferred directly to the device for storing the medical imaging data set.

Another advantage is that the specialist for analysing the medical imaging data sets can concentrate his focus on those medical imaging data sets having a reduced probability for a negative finding. Another advantage is concentrating on negative findings (instead of concentrating on positive findings), since in that way not all information being available for all potential abnormalities respectively have to be considered. Thus, comparing the medical imaging data sets to previous medical imaging data sets is simplified.

In at least one embodiment, the medical imaging data set is recorded by a medical imaging device such as a x-ray-scanner, a CT (computer tomography scanner)—scanner, a MRT (magnetic resonance tomography)—scanner or the like, and subsequently the medical imaging data set is provided, preferably to a control unit. The control unit comprises a processor being configured for executing at least:

providing the medical imaging data set automatically either
to an output device for analysing the medical imaging data set or
to a device for storing the medical imaging data set and/or for creating a report data set based on the probability value.

It is thinkable that the control unit is incorporated into a medical imaging device, a workstation, such as a personal computer, and/or a server or a system of servers, such as a network or a cloud. The medical imaging data set represents a three- or four-dimensional data set. Preferably, at least one of the steps according at least one embodiment of the invention is executed on a server, in particular a cloud.

The term “output device” is preferably generic for a device being configured for presenting or depicting a visualisation of the medical imaging data set. For example, the output device is a screen depicting a visualisation of the medical imaging data set or a printing device for printing a visualisation of the medical imaging data set, for example on a sheet. In other words: the output device presents the medical imaging data set in a suitable way for analysing the medical imaging data set in detail by an operator or a clinician.

The term “device for storing” preferably describes a memory device used for storing the recorded medical imaging data sets e.g. a digital storage medium such as a hard disc, SD-card, which may be part of a computer or cloud. Preferably, the medical imaging data sets being transferred directly to the device for storing by the control unit are labelled with a standard phrase for identifying them. Furthermore, it is thinkable that the output device and the device for storing the medical imaging data set are integrated into a common structure, such as a workstation, or the device for storing the medical imaging data sets is incorporated into a server or a system of servers, whereas the output device is included in a workstation or the medical imaging device.

In particular the report data set comprises a note or a text phrase being provided to the clinician in order to inform the clinician, whether the medical imaging data set has to be analysed in Detail or not. For example, the report data set is transferred by email.

Preferably, assigning the probability value to the medical imaging data set is realized by the control unit, in particular by pre-analysing the medical imaging data set. For example, the control unit compares the recorded medical imaging data set to previous recorded medical imaging data sets having either a negative finding or a positive finding. By specifying the type of abnormality the comparison can be limited to corresponding medical imaging data sets recorded in the past or to special parts of the medical imaging data set being relevant for the finding. As a consequence, a computing effort of the control unit can be reduced, since the control unit concentrates its pre-analysis or preselection to relevant medical imaging data set and/or relevant parts of the medical imaging data set.

Particularly advantageous embodiments and features of the invention are given by the dependent claims as revealed in the following description. Features of different claim categories may be combined as appropriate to give further embodiments not described herein.

According to a preferred embodiment, it is provided that an information data set is provided and wherein the probability value is based on the medical imaging data set and the information data set. By considering the information data set in addition to the medical imaging data set for determining the probability value, it is advantageously possible to further increase the success rate for having a positive finding in the detailed analysis by the specialist, since even more medical imaging data sets that need no detailed analysis can be sorted out. The term “information data set” preferably describes data sets that for example compile personal information of the patient such as an age, a sex of the patent, a lab result of the patient, patient record information, a first assessment and/or previous exams. In particular, the information data set is automatically extracted from a data base such as from a RIS (RadiologieInformationsSystem), a PACS (Picture Archiving and Communication System), a EMR (ErfahrungsMedizinischen Register), a HIS (Hospital Information System), a LIS (Labor-Informationssystem) or other. Preferably, the information data set is extracted from several data bases or constructed by several information extracted from different patient related data bases. It is also thinkable that the information data set is included in a label of the medical imaging data set and the control unit decodes the needed information from the labelling.

In particular, it is provided that the probability value is provided by a trained artificial network, wherein preferably the artificial network is trained by a machine leaning mechanism, in particular a deep learning mechanism, and/or by using a Siamese algorithm. Preferably, it is provided that a data driven machine learning artificial intelligence method is used for determining the probability value. For example, an algorithm framework uses a deep learning (conventional neural network) approach that learns a similarity metric form a labelled dataset. The training approach employs a discriminative loss function that drives the similarity metric to be high for similar pairs and small for different pairs of medical imaging data sets. The convolutional neuronal network or any other data driven model is optimized to project images into a lower dimensional space robust to irrelevant differences (such as noise, artifacts, exposure, non-discriminant anatomical features) and discriminant for diseases classes relevant for a specific exam including a normal patient class. Preferably, a Siamese algorithm can be used for the training of the similarity metric.

Further, it is thinkable that a sampling rate is adapted for computing and aggregating the similarity of the recorded medical imaging data set to those saved in a training data base. For example, the labels of the closest matches will determine the probability value as well as a final rule-out label. As a consequence, the problem is reduced to a two class classification problem to discriminate between normal and any of the diseased classes.

Preferably, the sampling is adjusted to the patient information, in particular to at least a part of the information training data set. Thus, it is possible to increase a success rate for comparing the recorded medical imaging data set to previous medical imaging data sets. In particular, an interrogation of individuals in the database of the same age, sex, race, BMI or other parameters that may help eliminating biases is performed.

Preferably, it is provided that a result data set is provided after analysing the medical imaging data set and the result data set is used for training the artificial network. Preferably, the result data set is transferred to and stored at a data base of the artificial network. Thus, the artificial network is trained continuously by using the result data sets. In particular, the result data set comprises the medical imaging data set, the information data set and preferably the result of the detailed analysis. Alternatively and/or additionally artificial result data sets are incorporated to the training data base for periodically updating the artificial network.

In another preferred embodiment, it is provided that analysing is supported by an analysing device for highlighting an abnormality. Thus, it is possible to use the highlighting of the abnormality for comparing the medical imaging data set being analysed in detail and subsequently stored in the training data base of the artificial network to the present medical imaging data set in order to provide the probability value. For example, an area of the highlighted region is stored together with the medical imaging data set. When the control unit identifies a potential relevant region in the medical imaging data set it is possible to compare the area and/or shape of the potential relevant region to the area and/or shape of the highlighted region of the medical imaging data sets stored in the training data base for providing a probability value. It is also thinkable that the highlighting represents anatomic landmarks and/or that the analysing device highlights identified abnormality during the pre-analysis of the control unit (i. e. the analysis device is incorporated into the control unit in such cases).

Furthermore, it is provided that the result data set and/or the medical imaging data set is transferred to a data base of a clinical decision support system. In general, such a clinical decision support system collects all information about the patient and automatically suggests for example a further treatment of the patient. In particular, it is provided that the medical imaging data set being analysed in detail and the medical imaging data sets being transferred without further analyses are stored in the data base of the clinical decision support system.

According to another embodiment of the present invention, it is provided that the probability value is compared to a threshold value. Thus, the control unit can determine whether the medical imaging data set has to be analysed in detail or can be transferred directly to the storage device. It is also thinkable that the threshold value is adaptable, in particular adaptable by the control unit. By adapting the threshold value it is advantageously possible to set the threshold value such that the probability of transferring a medical imaging data set including an abnormality directly to the device for storing the medical imaging data set is as low as possible.

In another preferred embodiment, it is provided that a further probability value is provided based on the information data set, in particular before recording the medical imaging data set, and wherein the medical imaging data set is only automatically provided either
to the output device for analysing the medical imaging data set or
to a device for storing the medical imaging data set and/or for creating a report data set based on the probability value, when the difference between the threshold value and the further probability value is smaller than a further threshold.

As a consequence, it is possible to transfer those medical imaging data sets that should analysed anyway directly to the output device. For example, the information data set comprises an information about a previous disease and the probability of a return of this disease, i. e. the abnormality caused by the disease, is comparatively high. In such cases a detailed analysis should performed anyway. By skipping the step of determining whether the medical imaging data set should be transferred to the output device or the device for storing the medical imaging data set the control unit is released.

In another embodiment, it is provided that the information data set is based on a patient related data base. Thus, further information of the patient can be took into account for determining the probability value. Furthermore, it is thinkable that a class of specialist is suggested by the control unit based on the probability value. Thus, it is possible to direct difficult medical imaging data sets to experienced specialists.

Preferably, the information data set and/or the threshold value are entered via an input device. Preferably, the input device is a human machine interface such as a touch screen or a key board. Thus, it is possible to enter the information data set manually and subsequently transferring the information data set to the control unit for providing the probability value and/or further probability value.

In another embodiment of the present invention, it is provided that the information data set and/or the threshold value are set automatically, in particular by using the trained artificial network. In particular, it is provided that the control unit and/or the trained artificial network access to the patient related data base such that the control unit and/or the artificial network can get additional information on demand. For example, the control unit and/or the trained artificial network need further information about the patient stored in the PACS for providing the probability value.

In an example, a pneumothorax is suspected by a clinician of an emergency department, in particular after a blunt trauma. The pneumothorax is one of the most common forms of thoracic abnormalities. For instance, the clinician evaluates a preliminary probability of a pneumothorax bigger than 10%. In order to confirm the suspected pneumothorax a chest radiography and/or a high resolution CT is performed. In particular, a supine anteroposterior (AP) chest radiograph is performed for identifying the presence of the pneumothorax, wherein such a test has a likelihood of being negative between 0.25 and 0.72. By using the control unit it is provided that the medical imaging data set is used for identifying medical imaging data sets that are normal with a high sensitive, resulting in high probability value, and/or a likelihood of being negative close to 0. Thereby, a Mahalanobis distance might be used to quantify the variation in similarity of the current recorded medical imaging data set compared to the mean of the normal distribution in the data base of the artificial network.

The threshold can be set depending on the standard deviation. For example, the threshold is set more than 1.5 times of the standard deviation away from a mean of probability values of the previous recorded medical imaging data sets. In such cases when the probability value of the current medical imaging data set is higher than the threshold value, the medical imaging data set is classified as “normal” and a standard report data set is generated. In such cases when probability value is smaller than the threshold the current medical imaging data set is redirected to the output device for being analysed by a specialist.

A standard report data file, for example an ASCII report data file, comprises the following string or phrase: “findings:

No pre-examination for correlation. Upper mediastinum is slim and located in the middle. Size of the heart within standards. Hilar structures are resolvable in typical way for vessels on both sides. No signs of congestion at the pulmonary venous. No pleural effusion. No indication for pneumatic infiltrates in regions of the pulmonary tracts being visible free from overlapping. No pulmonary nodules definable. No indication for pleural dehiscence. Parts of the axial skeleton being imaged seem to be unobtrusive.

estimation:

No indication for cardinal decompensation or pneumatic infiltrates.”

Another embodiment of the present invention is directed to a system for analysing a medical imaging data set comprising the medical instrument and a server, wherein the system is configured for:

recording the medical imaging data set by a medical imaging device;

providing a probability value for a negative finding, in particular for a negative finding of a specific type of abnormality, based on the medical imaging data set; and providing the medical imaging data set automatically either
to an output device for analysing the medical imaging data set or
to a device for storing the medical imaging data set based on the probability value and/or for creating a report data set based on the probability value.

In particular, at least one embodiment of the system comprises a control unit having a processor configured for executing at least one of the steps of at least one embodiment of the method described above.

Another embodiment of the present invention is directed to a computer program product for carrying out the steps of the method according to at least one embodiment of the present invention when the computer program product is loaded into a memory of a programmable device.

A further embodiment of the present invention is a computer-readable medium on which is stored a program elements that can be read and executed by a computer unit in order to perform steps of at least one embodiment of the method according to the present invention when the program elements are executed by the computer unit.

Preferably, the programmable device and/or the computer unit are incorporated into the system for analysing the medical imaging data set described above, in particular into the control unit.

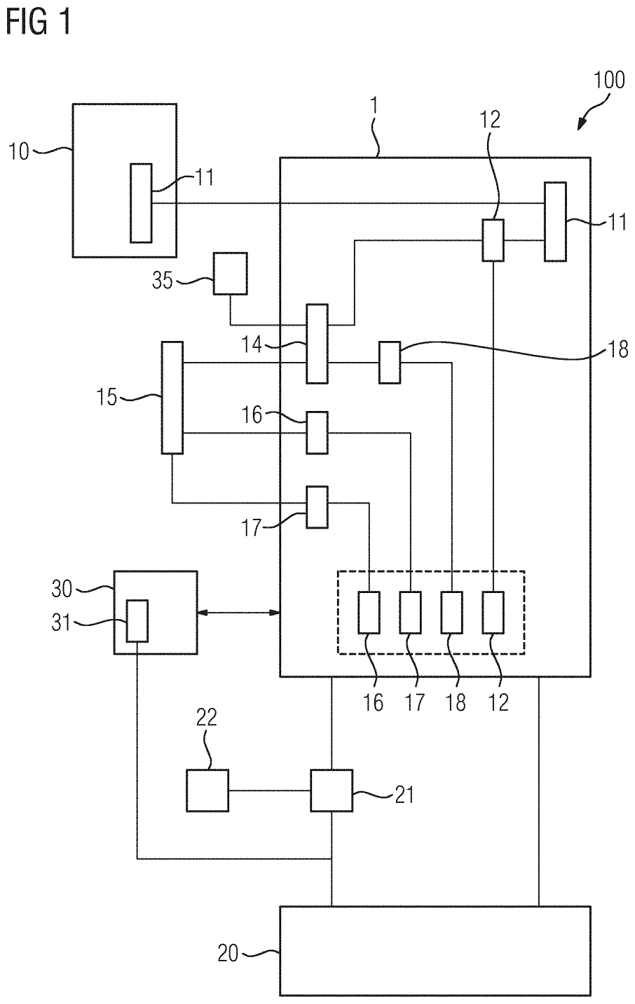

In FIG. 1 a block diagram illustrating a system 100 for analysing a medical imaging data set 11 according to a preferred embodiment of the present invention is shown. Such medical imaging data sets 11 are recorded and/or reconstructed by a medical imaging devices 10 such as a X-ray scanner, a computer tomography (CT) scanner, a magnetic resonance tomography (MRT) scanner or a ultrasound (US) scanner. Thus, it is possible to visualize inner parts of a patient for providing information that supports the clinical decision making process, i. e. a further treatment of the patient. For providing further relevant or needed information the medical imaging data set 10 is analysed by a specialist, such as a radiologist.

For optimizing the whole analyse procedure, in particular the workflow of analysing a plurality of medical imaging data sets, a control unit 1 for preselecting medical imaging data sets 11 is provided. The control unit 1 is configured for receiving the medical imaging data sets 11. In particular, the control unit 1 is part of a network, such as a cloud, or the control unit 2 is incorporated into a workstation, for example in a workstation of the medical imaging device, or into the medical imaging device 10. In the case of a control unit 2 being part of the network, the control unit 2 can receive medical imaging data sets 11 from different local medical imaging devices 10, for example located at different hospitals or different location within a hospital. In particular, the medical imaging device 10 and the control unit 2 are configured such that the medical imagine data set 11 is transferred to the control unit 1.

In particular, it is provided that a probability value 12 for a negative finding is assigned to the medical imaging data set 11, wherein the probability value 12 is based on the medical imaging data set 11. Preferably, for assigning a probability value 12 to the medical image data set 11 the control unit 1 comprises a trained artificial network 30 or is in communication with the artificial network 30, in particular an artificial network 30 trained by a machine learning mechanism such as a deep learning mechanism. As a consequence the control unit 1, in particular the trained artificial network 30, establishes a link between the medical imaging data set 11 and the probability value 12 for a negative finding, for example by identifying correlations between specific parameters of the medical imaging data set and the probability for identifying no abnormality by using the specific medical imaging device. In other words: By using the control unit 1, in particular the artificial network 30 and a training data base 31 of the artificial network, a preselection of the medical imaging data sets 11 is possible, wherein the medical imaging data sets 11 are ranked by their respective probability value 14 assigned or related to each medical imaging data set 11. Thereby a training data base 31 is included to the artificial network 30 and/or the artificial network 30 is in communication with the training data base 30.

Preferably, in addition to the medical imaging data set 11 an information data set 14 is transferred to the control unit 1. For example, the information data set 14 is added by entering information via an input device 15 and transferring the information data set 14 to the control unit 1. It is also thinkable that the information data set 14 is realized by extracting information from a patient related data base 35. For example, the control unit 1 extracts the information, in particular depending on the current medical imaging data set 11, from the patient related data base 35 automatically. Preferably, the information data set 11 comprises patient information such as an age of the patient, a sex of the patient, a medical record of the patient and/or a lab information. In particular, it is provided that the information data set is compiled automatically by accessing data from a RIS (radiological information system), a PACS (picture and communication system), a EMR (electronic medical record), a HIS (hospital information system), a LIS (laboratory information system) or comparable systems.

Further, the control unit 1 is configured for providing a probability value 12 based on the information data set 14 and/or the medical imaging data set 11. Thereby, the probability value 12 is individualized for each medical imaging data set 11 by assigning the probability value 12 to the medical imaging data set, in particular supported by the information data set 14 or by taking into account the information data set 14. For example, the probability value 12 is based on more than one, preferably more than three or even more than five parameter of the information data set 14 in addition to results of the pre-analysis of the medical imaging data set 11 by the control unit 1. It is also thinkable that the information data set 14 comprises a parameter specifying a disease or naming the disease. Another potential content of the information data set 11 might be an abnormality detected in the past.

Subsequently, the probability value 14 is compared to a threshold value 16. Based on this comparison, the control unit 1 automatically unit determines, whether the medical imaging data set 11 is transferred either

to an output device 21 for analysing the medical imaging data set 11 or

to a device for storing 20 the medical imaging data set 11 based on the probability value 12.

In particular, the medical imaging data set 11 is transferred to the output device 21 for analysing the medical imaging data set 11, when the probability value 12 is smaller than the threshold value 16. The output device 21 might be a display or a printer for visualising the medical imaging data set 11. In particular, the output device 21 present the medical imaging data set to a specialist. In other words: the control unit 1 decides, whether the medical imaging data set 11 is presented to a specialist, for example a radiologist, for further analysis. As a result, it is advantageously possible to skip these analyses of those medical imaging data sets 11 having a high probability for outputting a non-relevant result.

In particular, it is provided that the medical imagine data set 11 is directly transferred to a device 20 for storing the medical imaging data set 11, preferably device 20 for storing of a decision making support system, without further analysis, when the probability value 12 is greater than the threshold value 16, and is preferably further labelled with a standard phrase indicating that no further analysing of the medical imaging data 11 has been performed.

As a consequence, the total number of procedures over several analyses of different medical imaging data sets 11 can be reduced and therefore the system and/or the specialist for analysing the medical imaging data sets 11 is relieved.

It is also thinkable that a further probability value is provided based on the information data set 11, in particular before recording the medical imaging data set 11, and wherein the medical imaging data set 11 is only automatically provided either

to the output device 22 for analysing the medical imaging data set 11 or

to the device for storing 20 the medical imaging data set 11 based on the probability value 12, when the difference between the threshold value 14 and the further probability 18 value is smaller than a further threshold 17.

As a consequence, those medical imaging data sets 11 having a very low further probability are directly transferred to the output device 20 and preselecting by the control unit 1 is skipped. A very low further probability might be caused by a previous disease in the past. Thus, it is possible to make a preliminary selection for those medical imaging data sets 11 that definitively needs a detailed analysis or have such a high probability that there is no need for calculating the probability value 12 by the control unit 1. As a result, a transfer of those medical imaging data sets and information data sets 14, whose output of the control unit 1 is predictable, can be avoided, advantageously.

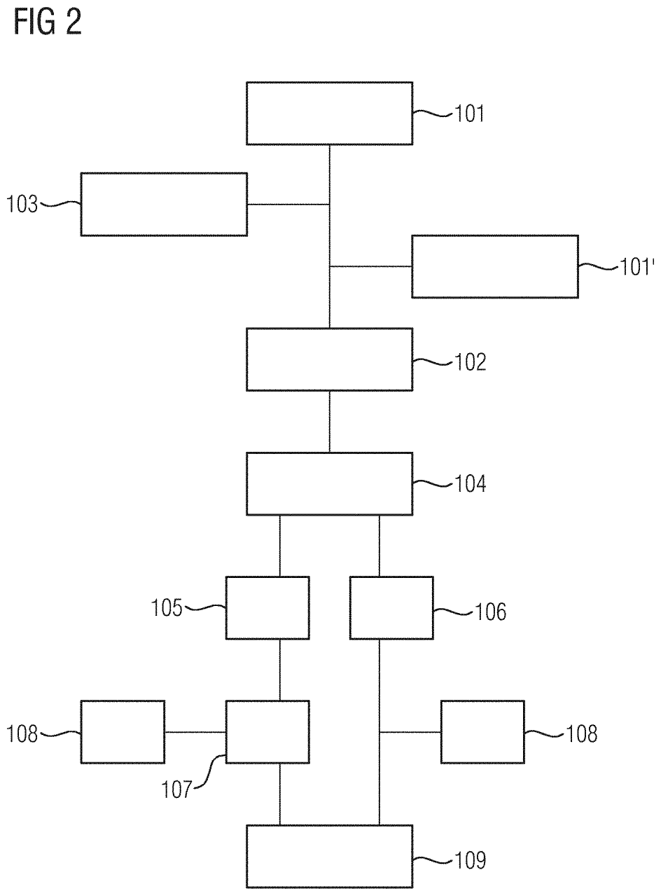

In FIG. 2 a flow diagram illustrating a method for analysing a medical imaging data set 11 according to a preferred embodiment of the present invention is illustrated. Thereby the method comprises:

providing 101 a medical imaging data set 11,

providing 101′ the information data set 14,

assigning 102 a probability value 12 to the medical imaging data set 11, in particular assigning 102 a probability value 12 based on the medical imaging data set 11 and/or the information data set 14 or assigning 102′ a further probability value 18 to the information data set 14,

configuring 103 the control unit 2, in particular by determining the threshold value 14 and the further threshold value 17,

comparing 104 the probability value 12 to the threshold value 16 or comparing the difference of the further probability value 18 and the threshold value 16 to the further threshold value 17

transferring 105 the medical imaging data set 11 to the output device 21 or transferring 106 the medical imaging data set 11 to the device 20 for storing the medical imaging data set 11 based on the probability value 14

analysing 107 the medical imaging data set 11, in particular at the output device 21,

training 108 the artificial network 30, in particular by the analysed medical imaging data sets 11, and

saving 109 the analysed medical imaging data set 11 to the device 20 for storing the medical imaging data sets.

1. A method for analysing a medical imaging data set, comprising:
providing the medical imaging data set;
assigning a probability value for a negative finding to the medical imaging data set, the probability value being based on the image data set; and
at least one of
providing the medical imaging data set automatically to at least one of
an output device for analysing the medical imaging data set or to a device for storing the medical imaging data set based on the probability value, and
creating a report data set
based on the probability value.

2. The method claim 1, wherein an information data set is provided and wherein the probability value is based on the medical imaging data set and the information data set.

3. The method of claim 1, wherein the probability value is provided by a trained artificial network.

4. The method of claim 3, wherein a result data set is provided after analysing the medical imaging data set and wherein the result data set is used for training the artificial network.

5. The method of claim 4, wherein the analysing is supported by an analysing device for highlighting an abnormality.

6. The method of claim 4, wherein the at least one of the result data set and the medical imaging data set is transferred to a data base of a clinical decision support system.

7. The method of claim 1, wherein the probability value is compared to a threshold value.

8. The method of claim 2, wherein a further probability value is provided based on the information data set, and wherein the medical imaging data set is only automatically provided either
to the output device for analysing the medical imaging data set or
a device for at least one of storing the medical imaging data set based on the probability value and creating a report data set based on the probability value,
upon a difference between a threshold value and the further probability value being smaller than a further threshold.

9. The method of claim 2, wherein the information data set is based on a patient related data base.

10. The method of claim 1, wherein the medical imaging data set is recorded by a medical imaging device.

11. The method of claim 2, wherein at least one of the information data set and a threshold value are entered via an input device.

12. The method of claim 2, wherein at least one of the information data set and a threshold value are set automatically.

13. A system for analysing a medical imaging data set, the system comprising:
a medical imaging device to record the medical imaging data set; and
at least one processor, configured to
provide a probability value for a negative finding; and
provide the medical imaging data set automatically either
an output device for analysing the medical imaging data set or
a device for at least one of storing the medical imaging data set based on the probability value and creating a report data set based on the probability value.

14. A non-transitory computer program product storing a computer program for carrying out the method claim 1 when the computer program product is loaded into a memory of a programmable device and executed by the programmable device.

15. A non-transitory computer-readable medium on storing program elements, be readable and executable by a computer unit to perform the method of claim 1 when the program elements are executed by the computer unit.

16. The method of claim 3, wherein the artificial network is trained by at least one of a machine leaning mechanism and a Siamese algorithm.

17. The method of claim 16, wherein the machine leaning mechanism is a deep learning mechanism.

18. The method of claim 2, wherein the probability value is provided by a trained artificial network.

19. The method of claim 18, wherein a result data set is provided after analysing the medical imaging data set and wherein the result data set is used for training the artificial network.

20. The method of claim 19, wherein the analysing is supported by an analysing device for highlighting an abnormality.

21. The method of claim 5, wherein the at least one of the result data set and the medical imaging data set is transferred to a data base of a clinical decision support system.

22. The method of claim 8, wherein a further probability value is provided based on the information data set before recording the medical imaging data set.

23. The method of claim 2, wherein at least one of the information data set and the threshold value are set automatically by using a trained artificial network.
